(19) United States
(12) Patent Application Publication
(10) Pub. No.: US 2008/0312279 A1
(43) Pub. Date: Dec. 18, 2008

(54) Compositions for Treating Fragile X and Other Disorders Methods of Use Thereof, and Screening for Compounds for Fragile X and Other Disorders

(76) Inventors: Stephen T. Warren, Atlanta, GA (US); Shuang Chang, Atlanta, GA (US); Peng Jin, Atlanta, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/091,549

(22) PCT Filed: Oct. 26, 2006
(86) PCT No.: PCT/US06/41759

§ 371 Date: Jun. 17, 2008

Related U.S. Application Data
(60) Provisional application No. 60/730,294, filed on Oct. 26, 2005.

(51) Int. Cl. A61K 31/4462; C40B 30/06; A61K 31/4178

(52) U.S. Cl. 514/315; 506/10; 514/397

(57) ABSTRACT

Methods of screening compounds, active compositions, pharmaceutical compositions, methods of treating disorders, methods of preventing disorders, and kits to screen a library of compounds, are disclosed.

### CROSS-REFERENCE TO RELATED APPLICATION

This application claims priority to copending U.S. provisional application entitled, “COMPOSITIONS FOR TREATING FRAGILE X AND OTHER DISORDERS METHODS OF USE THEREOF, AND SCREENING FOR COMPOUNDS FOR FRAGILE X AND OTHER DISORDERS,” having Ser. No. 60/730,294, filed Oct. 26, 2005, which is entirely incorporated herein by reference.

### TECHNICAL FIELD

The present disclosure is generally directed to methods of screening compounds, compositions for treating mental retardation, and methods of treating mental retardation.

### BACKGROUND

Fragile X syndrome is a common inherited form of mental retardation caused by the absence of fragile X mental retardation protein (FMRP). FMRP is a selective RNA-binding protein that forms a messenger ribonucleoprotein complex that associates with polyribosomes. FMRP can suppress translation of target transcripts and is thought to play a role in the regulation of local protein synthesis at the synapse. The absence of FMRP results in excess translation of target mRNAs, such as MAP1B in mice or its Drosophila ortholog, Futsch. Synaptic plasticity requires local protein synthesis of preexisting messages. Electrophysiological measures of plasticity in FMRP-deficient mice, such as mGluR1/5-induced long-term depression (LTD), are exaggerated, consistent with the overtranslation of LTD-required protein(s). Indeed, hippocampal mGluR5-induced LTD, which is sensitive to protein synthesis inhibitors in wild-type mice, is not sensitive to the same inhibitors in FMRP-deficient mice, suggesting the preexistence and constitutive expression of key protein(s). Thus, a model has been posited in which the loss of the translational suppression by FMRP results in excessive postsynaptic synthesis of proteins normally induced by group 1 mGluR signaling, and leads, in part, to the fragile X syndrome phenotype (mGluR hypothesis of fragile X syndrome).

### SUMMARY

Generally, aspects of the present disclosure are directed to methods of screening compounds, active compositions, pharmaceutical compositions, methods of treating disorders, methods of preventing disorders, kits to screen a library of compounds, and the like.

Embodiments of the present disclosure include a method of screening compounds, among others, that includes: providing a library of compounds; disposing each of the compounds of the library in a food with a lethal amount of glutamate to form a compound/food mixture for each compound; disposing each compound/food mixture in a container; disposing a plurality of fly embryos into each container, wherein each fly embryo includes a dFmr1 mutation, wherein the food is lethal to fly embryos including the dFmr1 mutation; measuring pupae formation and adult flies in each container, wherein pupae formation and adult flies occur in containers including a compound that rescues the fly embryos including the dFmr1 mutation; and selecting compounds of the library that produce at least one of: pupae formation, adult fly formation, and combinations thereof.

Embodiments of the present disclosure include a pharmaceutical composition, among others, that includes: an active composition in combination with a pharmaceutically acceptable carrier, wherein the active composition is present in a dosage level effective to treat a disorder, wherein the disorder is a FMRP-loss related disorder, wherein the active composition includes an active compound selected from: isopilocarpine nitrate, nipecotic acid, creatinine, ergonovine maleate, clomiphene citrate, GABA, MPEP, and combinations thereof.

Embodiments of the present disclosure include a method of treating a disorder, among others, that includes: administering to a host having the disorder an effective amount of an active compound, wherein the disorder is a FMRP-loss related disorder, wherein the active composition includes an active compound selected from: a compound of the isopilocarpine family of compounds, a compound of the nipecotic acid family of compounds, a compound of the creatinine family of compounds, a compound of the ergonovine maleate family of compounds, a compound of the clomiphene citrate family of compounds, a compound of the GABA family of compounds, a compound of the MPEP family of compounds, and combinations thereof.

Embodiments of the present disclosure include a method of preventing a disorder, among others, that includes: administering to a host having a risk of developing the disorder an effective amount of an active compound, wherein the disorder is a FMRP-loss related disorder, wherein the active composition includes an active compound selected from: a compound of the isopilocarpine family of compounds, a compound of the nipecotic acid family of compounds, a compound of the creatinine family of compounds, a compound of the ergonovine maleate family of compounds, a compound of the clomiphene citrate family of compounds, a compound of the GABA family of compounds, a compound of the MPEP family of compounds, and combinations thereof.

Embodiments of the present disclosure include a method of treating a disorder, among others, that includes: administering to a host having the disorder an effective amount of an active compound, wherein the disorder is a glutamate excitotoxity, wherein the active composition includes an active compound selected from: a compound of the isopilocarpine family of compounds, a compound of the nipecotic acid family of compounds, a compound of the creatinine family of compounds, a compound of the ergonovine maleate family of compounds, a compound of the clomiphene citrate family of compounds, a compound of the GABA family of compounds, a compound of the MPEP family of compounds, and combinations thereof.

Embodiments of the present disclosure include a method of preventing a disorder, among others, that includes: administering to a host having a risk of developing the disorder an effective amount of an active compound, wherein the disorder is a glutamate excitotoxity, wherein the active composition includes an active compound selected from: a compound of the isopilocarpine family of compounds, a compound of the nipecotic acid family of compounds, a compound of the creatinine family of compounds, a compound of the ergonovine maleate family of compounds, a compound of the clomiphene citrate family of compounds, a compound of the GABA family of compounds, a compound of the MPEP family of compounds, and combinations thereof.

Embodiments of the present disclosure include a kit to screen a library of compounds that includes: a plurality of fly embryos, wherein each fly embryo includes a dFmr1 mutation, wherein a food containing a lethal amount of glutamate is lethal to fly embryos including the dFmr1 mutation; and a set of directions for use to screen the library of compounds.

Other compositions, methods, features, and advantages of the present disclosure will be or become apparent to one with skill in the art upon examination of the following drawings and detailed description. It is intended that all such additional compositions, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1D illustrate the development of a novel function-based drug screen for fragile X syndrome. FIG. 1(A) illustrates that dfmr1³ null flies exhibit lethality when raised on a commercial food (CF). The table shows the number of progeny from crosses of heterozygous (w¹¹¹⁸, dfmr1³/TM6C—top or w¹¹¹⁸; dfmr1³/Kr: GFP, TM6C—bottom) flies raised on CF or laboratory-prepared food (LF) (P<0.001). FIG. 1(B) illustrates dfmr1-deficient flies raised on LF are sensitive to supplemented glutamate. Embryos of w¹¹¹⁸, dfmr1³ +/− and dfmr1³ −/− were allowed to develop on LF supplemented with additional glutamate. Relative viability was calculated within each genotype as the percentage of viable adult progeny raised on LF supplemented with glutamate compared to progeny raised on LF alone. (For w¹¹¹⁸ only: anything above 100% was indicated as 100%). (Error bar indicate SEM; *=P<0.005). FIG. 1(C) illustrates supplementing CF with MPEP (10 μM) improved dfmr1³ −/− viability. Embryos from dfmr1³ +/− crosses were allowed to develop on LF, CF, or CF+MPEP, respectively. Relative viability was obtained by comparing numbers of dfmr1³ −/− adult progeny raised on CF or CF+MPEP to those on LF. (Error bars indicate SEM; *=P<0.005). FIG. 1(D) illustrates a diagram of a novel function-based small molecule screen for compounds that can rescue the dfmr1-dependent lethality.

FIGS. 2A-2M illustrate drug treatments to rescue abnormal mushroom body (MB) structure in the dfmr1³ null fly brain. Brains were dissected from 0- to 2-day-old flies and immunostained with anti-Fas II antibody (ID4) and an Alexa-conjugated secondary antibody. The symmetrical α, β and γ MB neuronal lobes are indicated. FIG. 2(A) illustrates normal brain MB structure in wild-type flies. FIG. 2(B-G) illustrate various MB structural abnormalities seen in dfmr1³ null mutants raised on LF. Representative deficiencies are indicated by the arrows each figure. The abnormalities include: midline crossing by the β lobes; broken ends and misdirecting of the β lobes; over-branching or truncation of β lobes; missing of one α lobe. FIGS. 2(I-L) illustrates the drug treatments during development that rescued the MB structural abnormalities associated with dfmr1 mutation. The neuronal lobes of the MBs were labeled with anti-Fas II antibody and appeared normal in these brains. Drug dosages used here were the optimal dosages determined in the viability assay. FIG. 2(M) illustrates the percentage of dfmr1³ null flies with normal MB morphology after treatment with or without drugs. Three replicate drug treatments were performed and the results were averaged. Error bars indicate SEM over the three experiments (P<0.005). The total number of flies scored is listed for each treatment.

FIGS. 3A-3B illustrate the over-expression of Futsch protein associated with dfmr1 deficiency is reduced by isopilocarpine and MPEP treatments. FIG. 3(A) illustrates a representative Western blot showing Futsch expression in w¹¹¹⁸, dfmr1³ +/−, and dfmr1³ −/− head lysates. Flies were raised on LF with or without drug supplements using the effective dosages. Heads from 0- to 2-day old adult flies were used to prepare protein lysates. β-actin was used as a loading control. FIG. 3(B) illustrates the quantification of relative Futsch protein level from multiple Western blots using Kodak Imaging System software. The quantifications were based on duplicate Western analyses obtained from triplicate collections of brain lysates from two separate drug treatments (6 sets of samples total). The futsch levels were first normalized to β-actin levels, then plotted as the percentage of change of each treatment/genotype compared to w¹¹¹⁸ (no drug), which was set as 100%. MPEP, isopilocarpine treatment, w¹¹¹⁸ (no drug) and dfmr1³ +/− (no drug) showed significant difference compared with dfmr1³ −/− (no drug). Single factor ANOVA analysis showed significant differences between genotypes/treatments (P<0.05) and post-hoc t-test (two-samples assuming equal variances) were performed to determine significance compared to dfmr1³ −/− (no drug) (Error bars indicate SEM; *=P<0.05).

FIG. 4 illustrates excessive glutamate in CF. HPLC amino acid analyses were conducted on aqueous extracts of CF and LF food preparations. The peak at position 16.63 indicates glutamate. The CF contained 1.76-fold more glutamate than the LF.

FIG. 5 illustrates Table 1. Table 1 describes the top 15 compounds identified from the small molecule screen. Table 1 states drug names, percentage of pupae recovery from the initial screen, viability based on confirmation tests, optimized drug dosages, and general functional descriptions of the drugs. Three of the compounds involved in GABAergic pathway are shown in bold. The relative dfmr1 viability from the confirmation test was calculated as percentage of dfmr13 −/− among total flies raised on CF supplemented with drugs at the optimized dose compared to dfmr13 −/− recovered on LF only. (Optimized relative viability less than 20% was considered as false positive.)

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of synthetic organic chemistry, biochemistry, biology, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a compound” includes a plurality of compounds. In this specification and in the claims that follow, reference will be made to a number of terms that shall be defined to have the following meanings unless a contrary intention is apparent.

### DEFINITIONS

As used herein, the term “host” or “organism” includes both humans, mammals (e.g., cats, dogs, horses, etc.), and other living species that are in need of treatment. A living organism can be as simple as, for example, a single eukaryotic cell or as complex as a mammal. Hosts that are “predisposed” to neuronal disorders and related conditions can be defined as hosts that do not exhibit overt symptoms of one or more of these conditions but that are genetically, physiologically, or otherwise at risk of developing one or more of these conditions. Thus, compositions and agents of the present disclosure can be used prophylactically for these conditions. Further, a “composition” or “agent” can include one or more chemical compounds and/or agents, as described below. An “active composition” can include one or more “active compounds”.

The term “screening” refers to the identification of one or more compounds from a library of compounds that satisfy criteria such as, but not limited to, rescuing specifically designed flies from lethal dosages of glutamate. The screening methods of the present disclosure are used to identify compounds (e.g., drug candidates to be used in an active composition) for the treatment of disorders related to the loss of FMRP and other diseases and disorders as described herein. In addition, the screening methods of the present disclosure are used to identify compounds (e.g., drug candidates to be used in an active composition) for reversing, to at least some degree, glutamate excitotoxity (e.g., Parkinsons disease and/or the consequence of a stroke).

The term “derivative” means a modification to the disclosed compounds including but not limited to hydrolysis, reduction, or oxidation products of the disclosed compounds. In particular, the term encompasses opening of a nitrogen containing ring structure, including but not limited, to an imidazole of the disclosed compounds. Hydrolysis, reduction, and oxidation reactions are known in the art.

The term “therapeutically effective amount” as used herein refers to that amount of the compound being administered that will relieve to some extent one or more of the symptoms of the disorder being treated, and/or that amount that will prevent, to some extent, one or more of the symptoms of a disorder that the hose being treated is at risk of developing.

“Pharmaceutically acceptable salt” refers to those salts that retain the biological effectiveness and properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

A “pharmaceutical composition” refers to a mixture of one or more of the compounds described herein, or pharmaceutically acceptable salts thereof, with other chemical components, such as physiologically acceptable carriers and excipients. One purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11,:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenyloin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

As used herein, the term “topically active agents” refers to compositions of the present disclosure that elicit pharmacological responses at the site of application (contact in a topical application) to a host.

As used herein, the term “topically” refers to application of the compositions of the present disclosure to the surface of the skin and mucosal cells and tissues.

Throughout the specification, groups and substituents thereof may be chosen to provide stable moieties and compounds.

The disclosed compounds form salts that are also within the scope of this invention. Reference to a compound of any of the formulas herein is understood to include reference to salts thereof, unless otherwise indicated. The term “salt(s)”, as employed herein, denotes acidic and/or basic salts formed with inorganic and/or organic acids and bases. In addition, when an active compound of formula I contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds of an active compound may be formed, for example, by reacting an active compound with an amount of acid or base, such as an equivalent amount, in a medium such as one in which the salt precipitates or in an aqueous medium followed by lyophilization.

The disclosed compounds that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The disclosed compounds that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the disclosure are also contemplated herein. Solvates of the compounds are preferably hydrates.

To the extent that the disclosed active compounds, and salts thereof, may exist in their tautomeric form, all such tautomeric forms are contemplated herein as part of the present disclosure.

All stereoisomers of the present compounds, such as those which may exist due to asymmetric carbons on the various substituents, including enantiomeric forms (which may exist even in the absence of asymmetric carbons) and diastereomeric forms, are contemplated within the scope of this disclosure. Individual stereoisomers of the compounds of the disclosure may, for example, be substantially free of other isomers, or may be admixed, for example, as racemates or with all other, or other selected, stereoisomers. The chiral centers of the compounds of the present disclosure can have the S or R configuration as defined by the IUPAC 1974 Recommendations.

The terms “including”, “such as”, “for example” and the like are intended to refer to exemplary embodiments and not to limit the scope of the present disclosure.

### Discussion

Embodiments of the present disclosure include screening methods, kits for screening a library of compounds, active compositions including one or more active compounds, pharmaceutical compositions including one or more active compounds, methods of treating and/or preventing fragile X mental retardation protein (FMRP)-loss related disorders and related neural disorders, kits for treating and/or preventing FMRP-loss related disorders and related neural disorders, methods of treating and/or preventing glutamate excitotoxity and related disorders and diseases, and the like.

In general, embodiments of the present disclosure include high throughput methods of screening compounds (e.g., drug candidates) that may be used for the treatment of FMRP-loss related disorders and/or related neural disorders as well as glutamate excitotoxity, anxiety disorders, and disorders of memory. The screen is a fly-based (e.g., drosophila (fruit fly)) compound screen, where the efficacy of the compounds is determined by scoring the number of pupae formation or the emergence of adult flies under certain incubation conditions. Compounds that satisfy the screening process may be selected as active compounds that can be used in compositions and pharmaceutical compositions to treat hosts in need of such treatment with an effective amount of the active composition.

Embodiments of the screening method are advantageous because they provide the only known approach for screening for compounds that may reverse the clinical consequence of FMRP loss. Moreover, using Drosophila provides a screen of a complex organism that is much more likely to provide a useful compound than more simple screens, such as cell based screens. Moreover, the human FMR1 gene and the Drosophila dFmr1 gene are highly conserved, and Drosophila mutants, lacking FMRP, display learning abnormalities consistent with the human phenotype.

In general, embodiments of the present disclosure provide an active composition including one or more active compounds that can be used to treat and/or prevent FMRP-loss related disorders and neuronal disorders caused by the FMRP. For example, the active composition can be used to treat and/or prevent fragile X syndrome. In addition, the active composition can be used to treat and/or prevent other disorders such as, but not limited to, cognitive disorders, mental retardations, autism, attention deficit hyperactivity disorder, depressive disorder, and combinations thereof. Further, the active composition can be used to treat and/or prevent anxiety disorders and disorders of memory, including but not limited to, Alzheimer's disease. Furthermore, the active composition can be used to treat and/or prevent glutamate excitotoxity disorders and diseases such as, but not limited to, Parkinsons disease and the consequence of a stroke.

### Screening

Embodiments of the present disclosure include high throughput methods of screening compounds (e.g., drug candidates) that may be used for the treatment of FMRP-loss related disorders. In addition, embodiments of the present disclosure include high throughput methods of screening compounds (e.g., drug candidates) that may be used for the treatment of related neural disorders, glutamate excitotoxity, anxiety disorders, and disorders of memory.

In particular, embodiments of the present disclosure include methods of screening compounds using a drosophila (fruit fly) based compound screen to identify drug candidates for the treatment of FMRP-loss related disorders. The fly carrying the dFmr1 (fly gene that encodes the homologue of human FMRP) mutation exhibits neuronal and behavioral defects similar to those reported in fragile X mouse models and in human patients. It was found that flies with a homozygous dfmr1 mutation exhibit lethality on a food source high in glutamate (e.g., a commercial food source (JAZMIX)) but not on a food source with normal or low amounts of glutamate (e.g., JAZMIX contained about 2 times as much glutamate as another food source with did not produce lethality).

The homozygous embryos are placed into an appropriate container (e.g., a 96-well container) containing a food with a lethal amount of glutamate and with or without a compound from a library of drugs candidates. The embryos were incubated at certain conditions for a certain amount of time (e.g., at about 25° C. for about 10 days) to allow embryonic development. The efficacy of the drug candidate is determined by scoring the number of pupae formation or the emergence of adult flies after a certain time frame. Files or pupae that came out from food with a nonlethal amount of glutamate were used as positive control, and flies or pupae that came out from food with lethal amounts of glutamate without drugs were used as negative control.

For lethality, the amount of glutamate included in the food source should be greater than about 5 microM. In some embodiments, the amount of glutamine in the food source is greater than about 20 microM.

In addition, the flies are engineered to carry a marker (e.g., a GFP (green fluorescence protein) marker) so that all heterozygous dFmr1 embryos display a characteristic of the marker, whereas homozygous embryos do not. The marker allows easy separation of homozygous dFmr1 embryos from embryos of other genotypes using a flow-cytometry based embryo sorter, although other sorting techniques can be used as known in the art. It should be noted other markers or techniques could be used to differentiate among the embryos.

In particular, a drosophila (fruit fly) based compound screen was developed to identify drug candidates for the treatment of the loss of FMRP related disorders, where the flies have the GFP as the marker to separate heterozygous and homozygous embryos. Since the lethality of food having high levels of glutamate is specific to flies carrying homozygous dFmr1 mutation, embodiments of the present disclosure provide for screen assays to select compounds that regulate FMRP biological function. Embodiments of the present disclosure provide a screen for compounds that directly and indirectly regulate FMRP function and will rescue homozygous dFmr1 flies from food induced lethality.

In an embodiment, heterozygous dFmr1 flies were crossed with heterozygous flies carrying GFP/balancer. Embryos of F1 progenies were harvested and sorted either manually using a fluorescent microscope or automatically using a flow-cytometric embryo sorter.

The separated homozygous embryos are then transferred to a regular fly culture vials or a 96-well container (e.g., 20 embryos per well) containing food with a lethal amount of glutamate with or without the drug (e.g., 300 μl of pre-mixed food and the drug). The embryos were incubated at 25° C. for about 10 days to allow embryonic development. The formation of pupae or adult flies can be directly quantified by visual inspection. Efficacy of drugs is determined by scoring the number of pupae formation or the emergence of adult flies after 10 days. Additional details regarding the active compositions and disorders are described in the Examples below.

### Compositions

In general, the active compositions including one or more active compounds can be used to treat and/or prevent FMRP-loss related disorders such as, but not limited to, the fragile X syndrome, cognitive disorders, mental retardations, autism, attention deficit hyperactivity disorder, depressive disorder, anxiety-related disorders, memory-related disorders, glutamate excitotoxity, and the like.

The active composition can include one or more active compounds such as, but not limited to, members of the pilocarpine and isopilocarpine family of compounds, members of the nipecotic acid and isonipecotic acid family of compounds, members of the creatinine family of compounds, members of the ergonovine maleate family of compounds, members of the dienestrol family of compounds, members of the clomiphene citrate family of compounds, members of the GABA family of compounds, members of the kojic acid family of compounds, members of the aminobenztropine family of compounds, members of the MPEP family of compounds, and combinations thereof.

In particular, the pilocarpine and isopilocarpine family of compounds can include, but is not limited to, pilocarpine, isopilocarpine, pilocarpine hydrochloride, pilocarpine nitrate, isopilocarpine hydrochloride, isopilocarpine nitrate, derivatives of each, precursors thereof, and the like. In an embodiment, the active composition includes pilocarpine nitrate.

In particular, the nipecotic acid and isonipecotic acid family of compounds can include, but is not limited to, nipecotic acid, isonipecotic acid, derivatives of each, precursors thereof, and the like. In an embodiment, the active composition includes nipecotic acid.

In particular, the creatinine family of compounds can include, but is not limited to, creatinine, derivatives thereof, precursors thereof, and the like. In an embodiment, the active composition includes creatinine.

In particular, the ergonovine maleate family of compounds can include, but is not limited to, ergonovine maleate, derivatives thereof, precursors thereof, and the like. In an embodiment, the active composition includes ergonovine maleate.

In particular, the dienestrol family of compounds can include, but is not limited to, dienestrol, derivatives thereof, precursors thereof, and the like. In an embodiment, the active composition includes dienestrol.

In particular, the clomiphene citrate family of compounds can include, but is not limited to, clomiphene citrate, derivatives thereof, precursors thereof, and the like. In an embodiment, the active composition includes clomiphene citrate.

In particular, the GABA family of compounds can include, but is not limited to, GABA, derivatives thereof, precursors thereof, and the like. In an embodiment, the active composition includes GABA.

In particular, the kojic acid family of compounds can include, but is not limited to, kojic acid, derivatives thereof, precursors thereof, and the like. In an embodiment, the active composition includes kojic acid.

In particular, the aminobenztropine family of compounds can include, but is not limited to, aminobenztropine, derivatives thereof, precursors thereof, and the like. In an embodiment, the active composition includes aminobenztropine.

In particular, the MPEP family of compounds can include, but is not limited to, MPEP, derivatives thereof, precursors thereof, and the like. In an embodiment, the active composition includes MPEP.

In another embodiment, the active composition includes one or more of the following active compounds: isopilocarpine nitrate, nipecotic acid, creatinine, ergonovine maleate, dienestrol, clomiphene citrate, GABA, kojic acid, aminobenztropine, and MPEP. In another embodiment, the active composition includes one or more of the following: isopilocarpine nitrate, nipecotic acid, creatinine, ergonovine maleate, clomiphene citrate and GABA.

Where such forms exist, the active compounds of the active composition (e.g., members of the pilocarpine and isopilocarpine family of compounds, members of the nipecotic acid and isonipecotic acid family of compounds, members of the creatinine family of compounds, members of the ergonovine maleate family of compounds, members of the dienestrol family of compounds, members of the clomiphene citrate family of compounds, members of the GABA family of compounds, members of the kojic acid family of compounds, members of the aminobenztropine family of compounds, members of the MPEP family of compounds, and combinations thereof) can include analogues, compound homologues, compound isomers, or derivatives thereof, that can function in a similar biological manner as the active compounds of the active composition to treat and/or prevent fragile X syndrome and other disorders described herein including cognitive disorders, mental retardations, autism, attention deficit hyperactivity disorder, and depressive disorder and related conditions in a host. In addition, where such forms exist, the active compounds of the active composition can include pharmaceutically acceptable salts, esters, and prodrugs of the active compounds of the active composition described or referred to herein.

Based on embodiments of the present disclosure and the discussion in the Examples, a dosage regime for the active composition can be developed. In general, the starting dose of most Phase I clinical trials is based on preclinical testing, and is usually quite conservative. A standard measure of toxicity of a drug in preclinical testing is the percentage of animals (rodents) that die because of treatment. The dose at which 10% of the animals die is known as the LD₁₀, which has in the past often correlated with the maximal-tolerated dose (MTD) in humans, adjusted for body surface area. The adjustment for body surface area includes host factors such as, for example, surface area, weight, metabolism, tissue distribution, absorption rate, and excretion rate. Thus, the standard conservative starting dose is one tenth the murine LD₁₀, although it may be even lower if other species (i.e., dogs) were more sensitive to the drug. It is anticipated that a starting dose for the active composition in Phase I clinical trials in humans will be determined in this manner. This dosing regimen is discussed in more detail in Freireich E J, et al., Cancer Chemother Rep 50:219-244, 1966, which is incorporated herein by reference.

As stated above, a therapeutically effective dose level will depend on many factors. In addition, it is well within the skill of the art to start doses of the active composition at relatively low levels, and increase the dosage until the desired effect is achieved.

### Pharmaceutical Active Compositions

Embodiment of the present disclosure provide compositions and pharmaceutical compositions including the active composition (e.g., one or more active compounds) in an effective amount to treat and/or prevent a disorder such as those described herein.

Pharmaceutically active compositions and dosage forms of the disclosure include a pharmaceutically acceptable salt of disclosed or a pharmaceutically acceptable polymorph, solvate, hydrate, dehydrate, co-crystal, anhydrous, or amorphous form thereof. Specific salts of disclosed compounds include, but are not limited to, sodium, lithium, potassium salts, and hydrates thereof.

Pharmaceutical compositions and unit dosage forms of the disclosure typically also include one or more pharmaceutically acceptable excipients or diluents. Advantages provided by specific compounds of the disclosure, such as, but not limited to, increased solubility and/or enhanced flow, purity, or stability (e.g., hygroscopicity) characteristics can make them better suited for pharmaceutical formulation and/or administration to patients than the prior art.

Pharmaceutical unit dosage forms of the compounds of this disclosure are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure will typically vary depending on their use. For example, a dosage form used in the acute treatment of a disease or disorder may contain larger amounts of the active ingredient, for example the disclosed compounds or combinations thereof, than a dosage form used in the chronic treatment of the same disease or disorder. Similarly, a parenteral dosage form may contain smaller amounts of the active ingredient than an oral dosage form used to treat the same disease or disorder. These and other ways in which specific dosage forms encompassed by this disclosure will vary from one another will be readily apparent to those skilled in the art. See, e.g., Remington's Pharmaceutical Sciences, 18th ed., Mack Publishing, Easton, Pa. (1990).

Typical pharmaceutical compositions and dosage forms comprise one or more excipients. Suitable excipients are well known to those skilled in the art of pharmacy or pharmaceutics, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that comprise primary or secondary amines are particularly susceptible to such accelerated decomposition.

The disclosure further encompasses pharmaceutical compositions and dosage forms that include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate or organic acids. A specific solubility modulator is tartaric acid.

Like the amounts and types of excipients, the amounts and specific type of active ingredient in a dosage form may differ depending on factors such as, but not limited to, the route by which it is to be administered to patients. However, typical dosage forms of the compounds of the disclosure comprise a pharmaceutically acceptable salt, or a pharmaceutically acceptable polymorph, solvate, hydrate, dehydrate, co-crystal, anhydrous, or amorphous form thereof, in an amount of from about 10 mg to about 1000 mg, preferably in an amount of from about 25 mg to about 750 mg, and more preferably in an amount of from 50 mg to 500 mg.

Additionally, the compounds and/or compositions can be delivered using lipid- or polymer-based nanoparticles. For example, the nanoparticles can be designed to improve the pharmacological and therapeutic properties of drugs administered parenterally (Allen, T. M., Cullis, P. R. Drug delivery systems: entering the mainstream. Science. 303(5665): 1818-22 (2004)).

### Oral Dosage Forms

Pharmaceutical active compositions of the disclosure that are suitable for oral administration can be presented as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington's Pharmaceutical Sciences, 18th ed., Mack Publishing, Easton, Pa. (1990).

Typical oral dosage forms of the compositions of the disclosure are prepared by combining the pharmaceutically acceptable salt of disclosed compounds in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents.

Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent.

Examples of excipients that can be used in oral dosage forms of the disclosure include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the disclosure is typically present in from about 50 to about 99 weight percent of the pharmaceutical composition or dosage form.

Disintegrants are used in the compositions of the disclosure to provide tablets that disintegrate when exposed to an aqueous environment. Tablets that contain too much disintegrant may swell, crack, or disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the disclosure. The amount of disintegrant used varies based upon the type of formulation and mode of administration, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used to form pharmaceutical compositions and dosage forms of the disclosure include, but are not limited to, agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL® 200, manufactured by W. R. Grace Co. of Baltimore, Md.), a coagulated aerosol of synthetic silica (marketed by Degussa Co. of Plano, Tex.), CAB-O-SIL® (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

This disclosure further encompasses lactose-free pharmaceutical compositions and dosage forms, wherein such compositions preferably contain little, if any, lactose or other mono- or di-saccharides. As used herein, the term “lactose-free” means that the amount of lactose present, if any, is insufficient to substantially increase the degradation rate of an active ingredient.

Lactose-free compositions of the disclosure can comprise excipients which are well known in the art and are listed in the USP (XXI)/NF (XVI), which is incorporated herein by reference. In general, lactose-free compositions comprise a pharmaceutically acceptable salt of a compound in the active composition, a binder/filler, and a lubricant in pharmaceutically compatible and pharmaceutically acceptable amounts. Preferred lactose-free dosage forms comprise a pharmaceutically acceptable salt of the disclosed compounds, microcrystalline cellulose, pre-gelatinized starch, and magnesium stearate.

This disclosure further encompasses anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

### Controlled and Delayed Release Dosage Forms

Pharmaceutically acceptable salts of the disclosed active compounds can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Chemg-ju, Controlled Release Dosage Form Design, 2 (Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

One embodiment of the disclosure encompasses a unit dosage form that includes a pharmaceutically acceptable salt of the disclosed compounds (e.g., a sodium, potassium, or lithium salt), or a polymorph, solvate, hydrate, dehydrate, co-crystal, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the disclosure. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the disclosure include, but are not limited to, the OROS® Push-Pull™, Delayed Push-Pull™, Multi-Layer Push-Pull™, and Push-Stick™ Systems, all of which are well known. See, e.g., worldwide website alza.com. Additional OROS® systems that can be used for the controlled oral delivery of compounds and compositions of the disclosure include OROS®-CT and L-OROS®; see, Delivery Times, vol. 11, issue II (Alza Corporation).

Conventional OROS® oral dosage forms are made by compressing a drug powder (e.g., a salt of a compound of the active composition) into a hard tablet, coating the tablet with cellulose derivatives to form a semi-permeable membrane, and then drilling an orifice in the coating (e.g., with a laser). Kim, Chemg-ju, Controlled Release Dosage Form Design, 231-238 (Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively delivery drugs with low water solubility. Because salts of compound of the active composition and complexes of this disclosure (e.g., an compound sodium salt of the active composition) may be far more soluble in water than an active compound itself, they may be well suited for osmotic-based delivery to patients. This disclosure does, however, encompass the incorporation of an active compound, and non-salt isomers and isomeric mixtures thereof, into OROS® dosage forms.

A specific dosage form of the active compositions of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent the exit orifice and in direct or indirect contacting relationship with the expandable layer; and a flow-promoting layer interposed between the inner surface of the wall and at least the external surface of the drug layer located within the cavity, wherein the drug layer includes a salt of a compound of the active composition, or a polymorph, solvate, hydrate, dehydrate, co-crystal, anhydrous, or amorphous form thereof. See U.S. Pat. No. 6,368,626, the entirety of which is incorporated herein by reference.

Another specific dosage form of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a salt of a compound of the active compound, or a polymorph, solvate, hydrate, dehydrate, co-crystal, anhydrous, or amorphous form thereof. See U.S. Pat. No. 6,342,249, the entirety of which is incorporated herein by reference.

### Parenteral Dosage Forms

Parenteral dosage forms can be administered to patients by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Compounds that alter or modify the solubility of a pharmaceutically acceptable salt of an active composition disclosed herein can also be incorporated into the parenteral dosage forms of the disclosure, including conventional and controlled-release parenteral dosage forms.

### Topical, Transdermal and Mucosal Dosage Forms

Topical dosage forms of the disclosure include, but are not limited to, creams, lotions, ointments, gels, shampoos, sprays, aerosols, solutions, emulsions, and other forms known to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18.sup.th Ed., Mack Publishing, Easton, Pa. (1990).

Transdermal and mucosal dosage forms of the active compositions of the disclosure include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences, 18th Ed., Mack Publishing, Easton, Pa. (1990); and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer the active ingredient(s) of the disclosure include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466;465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with pharmaceutically acceptable salts of a compound of the active compositions of the disclosure. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, dehydrates, co-crystals, solvates, polymorphs, anhydrous, or amorphous forms of the pharmaceutically acceptable salt of a compound of the active composition can be used to further adjust the properties of the resulting composition.

### Kits

In some embodiments, active ingredients of the pharmaceutical compositions of the disclosure may not be administered to a patient at the same time or by the same route of administration. This disclosure therefore encompasses kits which, when used by the medical practitioner, can simplify the administration of appropriate amounts of active ingredients to a patient.

A typical kit includes a unit dosage form of a pharmaceutically acceptable salt of an active compound of the active composition. In particular, the pharmaceutically acceptable salt of an active compound of an active composition is the sodium, lithium, or potassium salt, or a polymorph, solvate, hydrate, dehydrate, co-crystal, anhydrous, or amorphous form thereof. A kit may further include a device that can be used to administer the active ingredient. Examples of such devices include, but are not limited to, syringes, drip bags, patches, and inhalers. The kit may include directions for use of the kit.

Kits of the disclosure can further include pharmaceutically acceptable vehicles that can be used to administer one or more active ingredients (e.g., an active compound). For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration.

Examples of pharmaceutically acceptable vehicles include, but are not limited to: water for injection USP; aqueous vehicles such as, but not limited to, sodium chloride injection, Ringer's injection, dextrose injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

In another embodiment, a kit for screening compounds for drug candidates includes a plurality of fly embryos. Each fly embryo includes a dFmr1 mutation (where a food containing a lethal amount of glutamate is lethal to fly embryos including the dFmr1 mutation) and a set of directions for use to screen a library of compounds.

### EXAMPLES

Now having described the embodiments of the present disclosure, in general, Examples 1 and 2 describe some additional embodiments of the present disclosure. While embodiments of the present disclosure are described in connection with Examples 1 and 2 and the corresponding text and figures, there is no intent to limit embodiments of the present disclosure to these descriptions. On the contrary, the intent is to cover all alternatives, modifications, and equivalents included within the spirit and scope of embodiments of the present disclosure.

### Example 1

A drug screen of 2000 compounds revealed 9 that rescue lethality of dfmr1-deficient flies raised on a high glutamate diet as well as rescuing additional phenotypes in the Drosophila model of fragile X syndrome.

Fragile X syndrome is caused by the functional loss of the fragile X mental retardation 1 (FMR1) gene. Excess glutamate excitatory signaling has been suggested as a consequence of FMR1 loss. It has been discovered that dfmr1-deficient Drosophila die when reared on a commercial food source, due to excess glutamate signaling. Using this phenotype, a 2,000 compound library was screened and identified at least 9 compounds that rescued the lethality, including at least 3 that implicate the GABAergic inhibitory pathway. Some of these compounds also rescued known dfmr1-deficient phenotypes, including mushroom body defects and excess Futsch translation. Such screens have substantial potential for therapeutic drug development for fragile X syndrome.

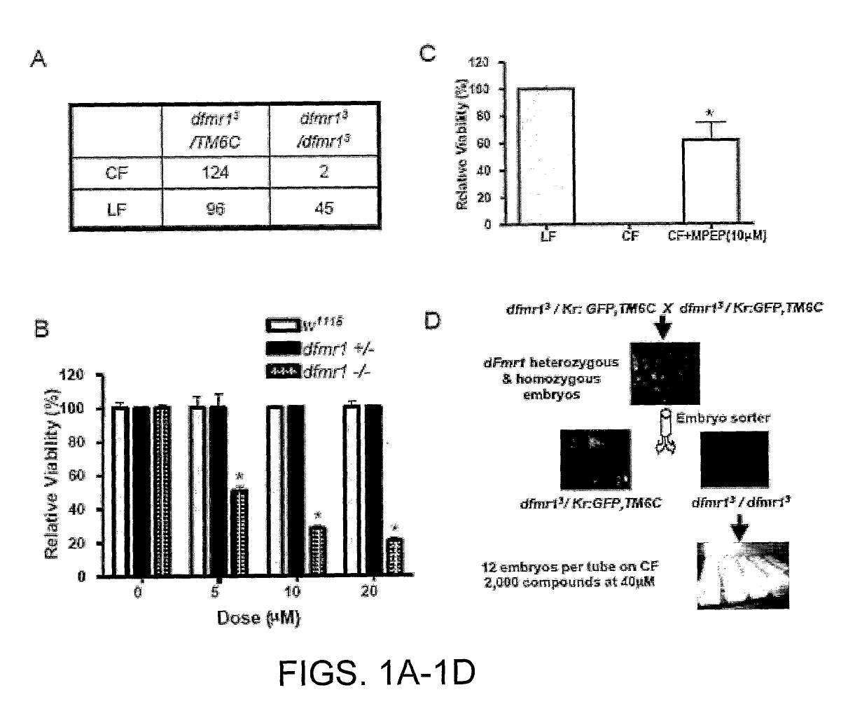

As mentioned above, it was discovered that dfmr1-deficient Drosophila exhibited lethality on Jazz-Mix™ (Applied Scientific) commercial food (CF), which did not occur when they were raised on laboratory-prepared food (LF). Lethality on CF was observed prior to puparium formation during development. A standard viability assay of adult progeny from crosses of heterozygous Drosophila (w¹¹¹⁸; dfmr1³/TM6C—dfmr1³ is a null allele) resulted in the expected 2:1 heterozygous to homozygous genetic ratios (94:47 expected; 96:45 observed) when the flies were grown on LF (FIG. 1A). In contrast, there was a marked absence of homozygous mutant progeny when the flies were raised on CF (62 expected; 2 observed; p<0.001).

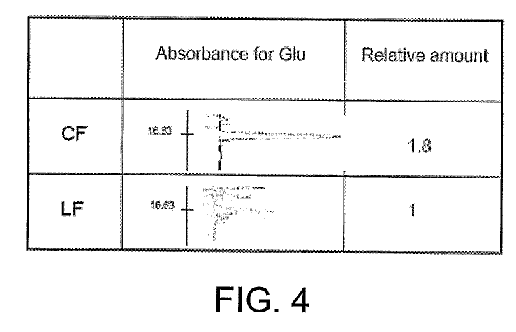

To determine the cause of the observed CF lethality of dfmr1-deficient flies, HPLC amino acid analyses was performed on aqueous extracts of CF and LF food preparations and noted that the CF contained 1.8-fold more glutamate than the LF (FIG. 4). Since this could result in excess glutamate signaling, exacerbating the consequences of dFmrp loss, it was next determined whether supplementing LF with additional glutamate could decrease the viability of dfmr1-deficient flies. As shown in FIG. 1B, when glutamate was added to LF, it was observed that a dose-dependent decline in viability of the dfmr1 homozygous null progeny (p<0.005). Since the mGluR5 antagonist MPEP is known to rescue other dfmr1-deficient phenotypes in the fly by antagonizing the Drosophila ortholog DmGluRA, these data would suggest that MPEP might also restore viability of dfmr1-deficient embryos raised on CF. Indeed, 10 μM MPEP added to the CF rescued the homozygous deficient flies to approximately 60% of their normal viability on LF (FIG. 1C). These combined data suggest that the loss of viability of the dfmr1 null flies raised on CF is due to a toxic effect of excess glutamate, consistent with the mGluR theory of fragile X syndrome.

Since the rescue of dfmr1 null lethality is an easily scored phenotype, a relatively high throughput strategy was designed to screen chemical libraries for small molecules that can restore the viability of dfmr1 null flies raised on CF (FIG. 1D). In order to seed test vials with dfmr1 null embryos only, the dfmr1 mutation was balanced with a GFP-TM6C chromosome (w¹¹¹⁸; dfmr1³/Kr: GFP, TM6C). This allowed sorting of the null dfmr1 mutants from other embryos by the absence of GFP fluorescence using a flow cytometry embryo sorter (COPAS SELECT). Twelve null embryos were automatically dispensed into each testing vial containing CF supplemented with 40 μM of individual compounds from a library of 2,000 drugs and natural products (The Spectrum Collection™, MicroSource Discovery Systems, Inc). Vials were then kept at 25° C. for 10 to 15 days to score for viability.

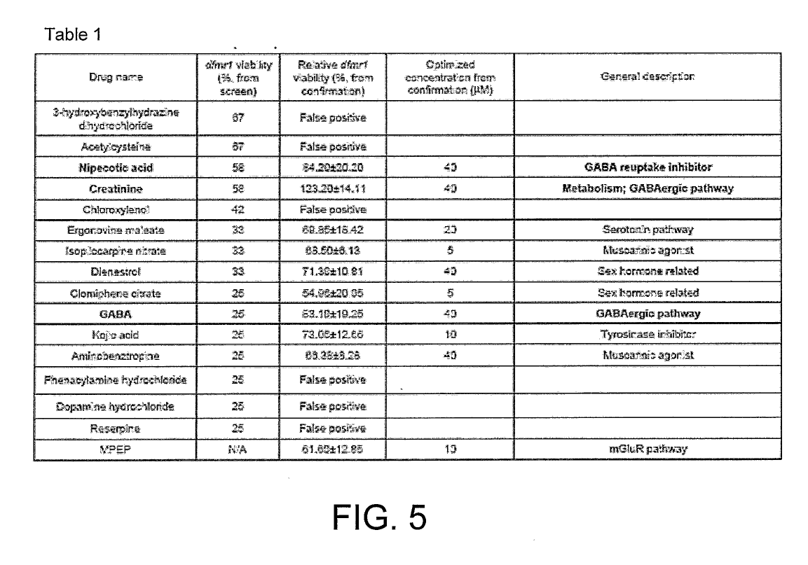

Among the 2,000 compounds screened, 61 were found to result in puparium formation or the emergence of adult flies. Fifteen of those compounds, as well as the MPEP-positive control, recovered pupal development from at least 25% of the seeded embryos (FIG. 5, Table 1). Each of these top 15 compounds underwent further validation using a larger-scale viability assay. Each vial was seeded with 100 embryos from dfmr1 heterozygous crosses, and adult progeny of both homozygous and heterozygous genotypes were counted 10 days later. Nine of the 15 compounds were validated and, for each of these, a dose-response analysis was performed to optimize the dosage (FIG. 5, Table 1). Using the optimal viability dose for each validated compound, it was explored whether other established phenotypes of dfmr1 deficiency are influenced by a selection of these compounds.

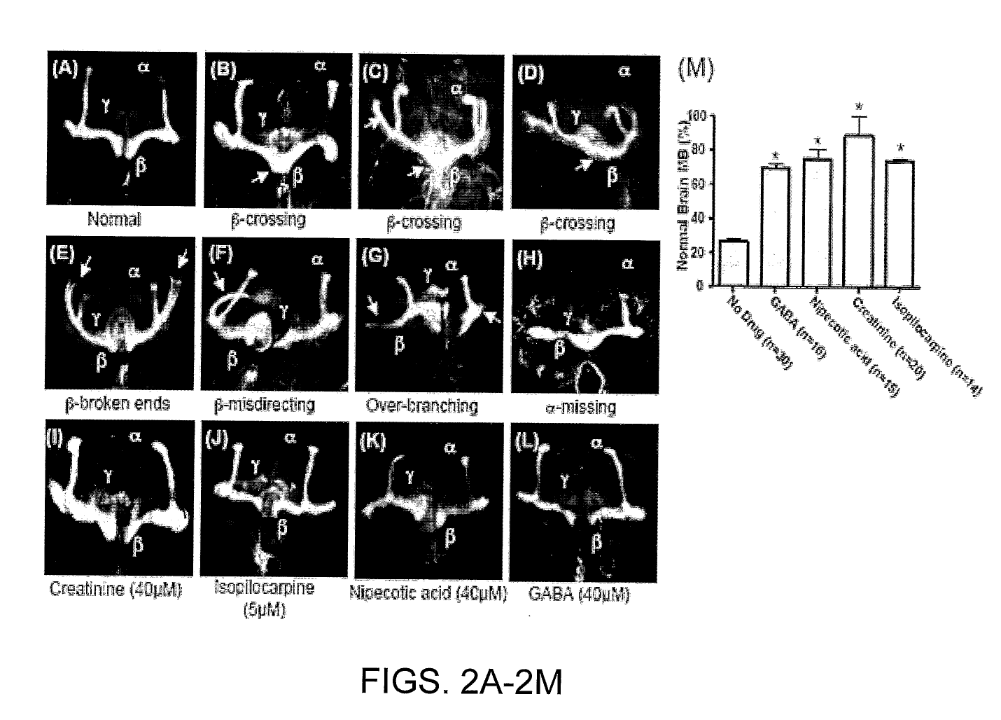

A striking morphological phenotype of dfmr1 deficiency in Drosophila involves structural abnormalities of the mushroom body in the fly brain. The mushroom body, important for Drosophila learning and memory, is an axon-like fiber structure composed of three paired neuronal lobes (the α, β, and γ lobes). In dfmr1 null flies, the β lobes, which normally terminate at the midline cleft, often cross through the midline. In addition, dfmr1 null flies also have missing or misdirected a and β lobes, as well as truncated or over-branched lobes. Immunostaining of the mushroom body confirmed that only 24% of the dfmr1-deficient flies reared on LF alone showed normal mushroom body structure, while the rest showed a variety of defects (FIG. 2B-G, M), consistent with previous findings. To test whether compounds identified in the viability screen could rescue the mushroom body morphology, 0- to 2-day-old dfmr1 mutant adults were examined that developed on LF supplemented with four of the nine compounds identified above (GABA, nipecotic acid, creatinine, and isopilocarpine). As shown in FIG. 2H-M, it was found that all four compounds significantly improved the mushroom body defects, restoring normal mushroom body morphology in 69-88% of null flies. This is comparable to the morphology rescued by MPEP. Thus, all four compounds tested that were identified in the viability screen also substantially improved the mushroom body defects associated with dfmr1 deficiency.

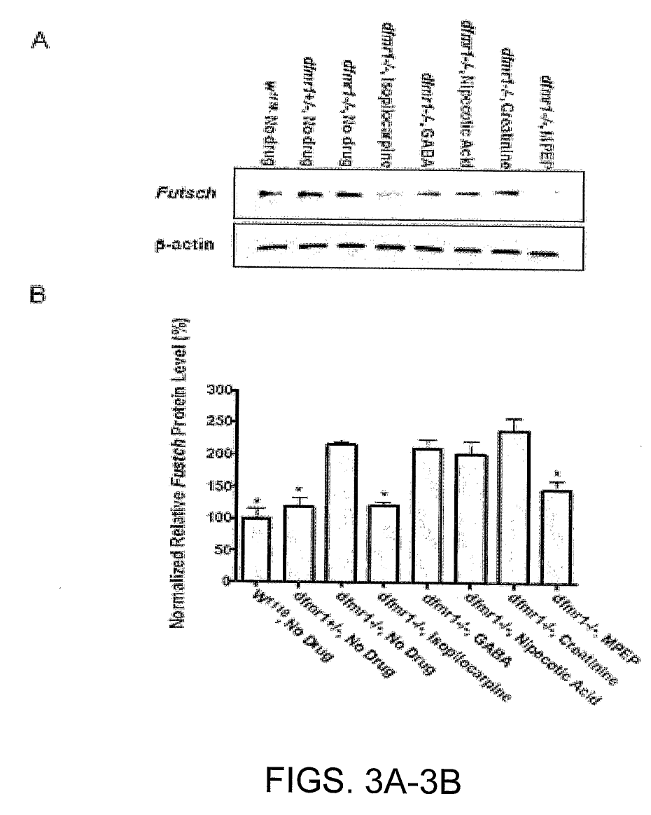

Additionally, it was tested whether the same four compounds could rescue the biochemical phenotype of Futsch over-expression that is associated with dfmr1 deficiency. The mRNA of Futsch is normally bound and translationally repressed by dFmrp. Thus, loss of dFmrp results in excess Futsch translation. As shown in FIG. 3, lysates from fly heads replicate this phenotype showing elevated Futsch protein level in dfmr1 homozygous null flies compared to wild-type (˜2-fold increase). The mGluR antagonist MPEP significantly reduced the level of Futsch in the null flies and isopilocarpine completely rescued this phenotype of the deficient flies. However, the three other compounds (GABA, nipectic acid and creatinine) that rescued both the CF lethality and the mushroom body defects did not significantly influence the elevated Futsch levels. These data suggest that GABA, nipecotic acid, and creatinine may either act at a point downstream of Futsch translation or influence pathways distinct from those acted upon by either MPEP or isopilocarpine for this particular dfmr1 deficiency phenotype.

The additional analyses of these four compounds identified in the function-based screen confirm their influence on the morphological and biochemical phenotypes of the Drosophila model of fragile X syndrome. The predicted biological functions of all nine of the confirmed drugs identified in the lethality screen implicate varying mechanisms of action, including GABAergic, serotonin, muscarinic and hormone related pathways and agents (FIG. 5, Table 1). Interestingly, three of the top compounds (GABA, nipecotic acid, and creatinine) identified in the lethality screen, have been implicated in the GABA inhibitory pathway. In addition to the natural agonist GABA, nipecotic acid is a GABA re-uptake inhibitor and creatinine is a condensation byproduct of creatine, which is involved in a pathway that activates the GABA_{A} receptor. This is a cogent, albeit infrequent, outcome of a chemical library screen where the top compounds identified make biological sense. Because excess signaling of a common synaptic excitatory pathway is believed responsible, in part, for fragile X syndrome, the identification of chemicals capable of enhancing the parallel inhibitory pathway is a plausible outcome of our screen. Several lines of evidence have implicated GABA in fragile X syndrome. Reduced expression of the mRNA for the GABA_{A} receptor subunit δ has been reported in the Fmr1 knockout mice as has a reduction in GABA_{A} receptor β subunit immunoreactivity in the cortex, diencephalons, and brainstem of adult fragile X mice. Moreover, it was shown that bicuculline, a GABA_{A} receptor antagonist, further reduced the lower dendritic spine density observed in Fmr1 knockout mice. Thus GABA agonists, whether compensating for reduced GABA_{A} receptor function or tempering excess excitatory stimulation by mGluR, may be a pharmacologically important addition to mGluR antagonists as potential therapeutic interventions for fragile X syndrome.

Materials and methods

### Drosophila Strains

Drosophila dfmr1 null mutant dfmr13/TM6C was published previously (T. C. Dockendorff et al., Neuron 34, 973 (2002), which is incorporated herein by reference). The Kr: GFP chromosome balancer fly was obtained from the Bloomington Drosophila Stock Center. Flies were maintained using standard procedures at 25° C. in 50%-70% humidity, on cornmeal-sucrose-yeast laboratory food (LF) that was supplemented with the mold inhibitor methylparaben and autoclaved. Recipe for making 10 litre LF: 14,000 ml water, 1,000 ml unsulfured molasses, 148 g agar, 1,000 ml cornmeal, 412 g baker's yeast, 225 ml tegosept (10% methyl p-hydrobenzoate in 95% ethanol) and 80 ml propionic acid. J azzmix food (CF) is purchased from Applied Scientific (Cat # AS 153) and is prepared following manufacturer's instruction.

### Drug Screen and Confirmation Procedures:

### Fly Embryo Sorting

Flies carrying the dfmr13 mutation over a GFP-balancer chromosome (Kr: GFP, TM6C) was necessary for embryo sorting. The GFP-balancer chromosome on LF resulted in lower segregation ratios, likely due to genetic background differences; however, the CF lethality remained. Embryos from crosses of heterozygous dfmr13/Kr. GFP, TM6C were collected and allowed to mature until they were at least six hours old to ensure GFP expression. Embryos were separated using the flow cytometry instrument COPAS SELECT (Union Biometrica) following manufacturer's instruction. Sorting accuracy is 99.7% based on manual verification of the first batch of embryos. Embryos were automatically placed into each testing vial of a 96 well plate containing CF and the candidate compounds.

### Drug Preparation

The Spectrum Collection™ compound library was obtained from MicroSource Discovery Systems, Inc. Drugs used in larger quantities for confirmation and subsequent analysis were obtained from Sigma (St. Louis, Mo.) or Tocris-Cookson (UK). Drugs supplemented in CF were added to the final concentration of 40 μM. LF was heated to liquefy food and allowed to cool briefly before drugs were added and mixed. In all cases 10 μl of green food coloring was added simultaneously with drug and used to control for uniform drug dispersion in the food.

### Fly Viability Assay

For the initial screen, 12 dfmr13/dfmr13 (GFP-) embryos were placed in each well of a 96 well plate with CF+ 40 μM of individual drugs. 96-well plates were kept at 25° C. and the percentage of pupae and adults recovered was scored. For confirmation of the top 15 drugs from the initial screen, viability assays were preformed starting about 100 embryos placed in regular fly culture vials from crosses of heterozygous dfmr13/Kr: GFP, TM6C flies. Adult progeny were counted and the relative viability of homozygous null flies was scored as the percentage of viable adult progeny raised on CF+ drug compared to the progeny on LF alone.

### Staining and Analysis of Mushroom Body Morphology

Brains from 0- to-2-day-old adults were dissected, fixed, and stained. Anti-Fascicilin II (ID4, Developmental Studies Hybridoma Bank) was used at 1:10 dilution and Alexa anti-mouse secondary antibody (Molecular probes) 1:100. Confocal microscopy was performed using a Leica Scanning laser confocal microscope. Neuronal lobe abnormalities were analyzed and scored by obtaining optical stacks of the [symbol] and β lobes as described.

### Quantitative Western Analyses

Quantitative Western analyses were performed. Briefly, 0- to 2-day-old adult Drosophila heads were obtained, and homogenized in lysis buffer (10 mM Tris (pH 7.5), 150 mM NaCl, 30 mM EDTA (pH 8.0), 0.5% Triton X-100, and 1× complete protease inhibitor (Boehringer Mannheim). 30 fly heads were collected per sample. 10 μg total protein lysates were subjected to SDS-PAGE electrophoresis on a 4%-20% gradient Tris-HCl gel (Bio-Rad). Monoclonal anti-22C10 against Futsch (1:500) was purchased from the Developmental Studies Hybridoma Bank (DSHB), University of Iowa. For the loading control, an antibody against Drosophila □-actin (Abeam) was used at a final concentration of 0.8 μg/ml. The signals were quantified using the Kodak Imaging System software.

### Example 2

### A: Pilocarpine Nitrate

It was found that supplementing 5 microM of Pilocarpine nitrate in JAZMIX food yielded 88.5% recovery of homozygous dFmr1 flies compared with 0% on JAZMIX food atone. In addition, Pilocarpine nitrate-treated dFmr1 filed demonstrated significant suppression of brain structural abnormality referred to as mushroom body formation. Mushroom body is critically involved in learning and memory, and was shown to be abnormal in dFmr1 flies. The treatment of Pilocarpine nitrate increased the percentage of normal brains from 27%- to 79%. Based on these novel findings, it can be concluded that Pilocarpine nitrate plays an important role in regulating FMRP-loss related biological function. This indicates that Pilocarpine nitrate may be used for the treatment of fragile X syndrome and other disorders including cognitive disorders, mental retardation, autism, attention deficit hyperactivity disorder, and depressive disorder.

### B: Nipecotic Acid

It was found that supplementing 40 microM of Nipecotic acid in JAZMIX food yielded 84% recovery of homozygous dFmr1 flies compared with 0% on JAZMIX food alone in addition, Nipecotic acid-treated dFmr1 flied demonstrated significant suppression of brain structural abnormality referred to as mushroom body formation. Mushroom body is critically involved in learning and memory, and was shown to be abnormal in dFmr1 flies. The treatment of Nipecotic acid increased the percentage of normal brains from 27% to 73%. Base on these novel findings, it was concluded that Nipecotic acid plays an important role in regulating FMRP-loss related biological function. This indicates that nipecotic acid may be used for the treatment of fragile X syndrome and other disorders including cognitive disorders, mental retardations, autism, attention deficit hyperactivity disorder, and depressive disorder.

### C: Creatinine

It was found that supplementing 40 microM of Creatinine in JAZMIX food yielded 123.2% recovery of homozygous dFmr1 flies compared with 0% on JAZMIX food alone. In addition, Creatinine-treated dFmr1 flies demonstrated significant suppression of brain structural abnormality referred to as mushroom body formation. Mushroom body is critically involved in learning and memory and was shown to be abnormal in dFmr1 flies. The treatment of Creatinine increased the percentage of normal brains from 27% to 85%. Based on these novel findings, it is concluded that creatinine plays an important role in regulating FMRP-loss related biological function. This indicates that Creatinine could be used for the treatment of fragile X syndrome and other disorders including cognitive mental retardations, autism attention deficit hyperactivity disorder, and depressive disorder.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and protected by the following claims.

Therefore, the following is claimed:

1. A method of screening compounds, comprising:
providing a library of compounds;
disposing each of the compounds of the library in a food with a lethal amount of glutamate to form a compound/food mixture for each compound;
disposing each compound/food mixture in a container;
disposing a plurality of fly embryos into each container, wherein each fly embryo includes a dFmr1 mutation, wherein the food is lethal to fly embryos including the dFmr1 mutation;
measuring pupae formation and adult flies in each container, wherein pupae formation and adult flies occur in containers including a compound that rescues the fly embryos including the dFmr1 mutation; and
selecting compounds of the library that produce at least one of: pupae formation, adult fly formation, and combinations thereof.

2. The method of claim 1, wherein the fly embryo is a homozygous embryo.

3. The method of claim 1, wherein the homozygous embryo is a fruit fly homozygous embryo.

4. A pharmaceutical composition comprising an active composition in combination with a pharmaceutically acceptable carrier, wherein the active composition is present in a dosage level effective to treat a disorder, wherein the disorder is a FMRP-loss related disorder, wherein the active composition includes an active compound selected from: isopilocarpine nitrate, nipecotic acid, creatinine, ergonovine maleate, clomiphene citrate, GABA, and combinations thereof.

5. The pharmaceutical composition of claim 4, wherein the FMRP-loss related disorder is selected from: a fragile X syndrome, a cognitive disorder, a mental retardation, autism, an attention deficit hyperactivity disorder, a depressive disorder, an anxiety disorder, a disorder of memory, and combinations thereof.

6. A pharmaceutical composition comprising an active composition in combination with a pharmaceutically acceptable carrier, wherein the active composition is present in a dosage level effective to treat a disorder, wherein the disorder is a FMRP-loss related disorder, wherein the active composition includes an active compound selected from: a compound of the isopilocarpine family of compounds, a compound of the nipecotic acid family of compounds, a compound of the creatinine family of compounds, a compound of the ergonovine maleate family of compounds, a compound of the clomiphene citrate family of compounds, a compound of the GABA family of compounds, and combinations thereof.

7. The pharmaceutical composition of claim 6, wherein the FMRP-loss related disorder is selected from: a fragile X syndrome, a cognitive disorder, a mental retardation, autism, an attention deficit hyperactivity disorder, a depressive disorder, an anxiety disorder, a disorder of memory, and combinations thereof.

8. A method of treating a disorder, comprising: administering to a host having the disorder an effective amount of an active compound, wherein the disorder is a FMRP-loss related disorder, wherein the active composition includes an active compound selected from: a compound of the isopilocarpine family of compounds, a compound of the nipecotic acid family of compounds, a compound of the creatinine family of compounds, a compound of the ergonovine maleate family of compounds, a compound of the clomiphene citrate family of compounds, a compound of the GABA family of compounds, and combinations thereof.

9. The method of claim 8, wherein the FMRP-loss related disorder is selected from: a fragile X syndrome, a cognitive disorder, a mental retardation, autism, an attention deficit hyperactivity disorder, a depressive disorder, an anxiety disorder, a disorder of memory, and combinations thereof.

10. The method of claim 8, wherein the active composition is selected from: isopilocarpine nitrate, nipecotic acid, creatinine, ergonovine maleate, clomiphene citrate, GABA, and combinations thereof.

11. A method of preventing a disorder, comprising: administering to a host at risk of developing the disorder an effective amount of an active compound, wherein the disorder is a FMRP-loss related disorder, wherein the active composition includes an active compound selected from: a compound of the isopilocarpine family of compounds, a compound of the nipecotic acid family of compounds, a compound of the creatinine family of compounds, a compound of the ergonovine maleate family of compounds, a compound of the clomiphene citrate family of compounds, a compound of the GABA family of compounds, and combinations thereof.

12. The method of claim 11, wherein the FMRP-loss related disorder is selected from: a fragile X syndrome, a cognitive disorder, a mental retardation, autism, an attention deficit hyperactivity disorder, a depressive disorder, an anxiety disorder, a disorder of memory, and combinations thereof.

13. The method of claim 12, wherein the active composition is selected from: isopilocarpine nitrate, nipecotic acid, creatinine, ergonovine maleate, clomiphene citrate, GABA, and combinations thereof.

14. A method of treating a disorder, comprising: administering to a host having the disorder an effective amount of an active compound, wherein the disorder is a glutamate excitotoxity, wherein the active composition includes an active compound selected from: a compound of the isopilocarpine family of compounds, a compound of the nipecotic acid family of compounds, a compound of the creatinine family of compounds, a compound of the ergonovine maleate family of compounds, a compound of the clomiphene citrate family of compounds, a compound of the GABA family of compounds, and combinations thereof.

15. A method of preventing a disorder, comprising: administering to a host at risk of developing the disorder an effective amount of an active compound, wherein the disorder is a glutamate excitotoxity, wherein the active composition includes an active compounds selected from: a compound of the isopilocarpine family of compounds, a compound of the nipecotic acid family of compounds, a compound of the creatinine family of compounds, a compound of the ergonovine maleate family of compounds, a compound of the clomiphene citrate family of compounds, a compound of the GABA family of and combinations thereof.

16. A kit to screen a library of compounds, comprising:
a plurality of fly embryos, wherein each fly embryo includes a dFmr1 mutation, wherein a food containing a lethal amount of glutamate is lethal to fly embryos including the dFmr1 mutation; and
a set of directions for use to screen the library of compounds.
